(19) United States
(12) Patent Application Publication
(10) Pub. No.: US 2013/0172823 A1
(43) Pub. Date: Jul. 4, 2013

(54) System for Delivery of Fluids and Use Thereof

(71) Applicant: Moti Meron, Herzliah (IL)

(72) Inventor: Moti Meron, Herzliah (IL)

(21) Appl. No.: 13/722,501

(22) Filed: Dec. 20, 2012

Related U.S. Application Data
(60) Provisional application No. 61/582,532, filed on Jan. 3, 2012.

(51) Int. Cl. A61M 5/19

(52) U.S. Cl. CPC A61M 5/19
USPC 604/205; 604/228; 604/236; 604/506

(57) ABSTRACT

Embodiments of the invention relate to a multi-syringe device where a coupling element causes a plurality of plungers to slide in unison within their respective barrels to simultaneously dispense fluids therefrom. At least one of the plungers is a variable-length plunger that is selectively engagable to the coupling element.

### FIELD OF THE INVENTION

Embodiments of the present invention relate to syringe-based system and related methods of use.

### BACKGROUND AND RELATED ART

Embodiments of the present invention relate to syringe-based system and related methods of use.

The following issued patents and patent publications provide potentially relevant background material, and are all incorporated by reference in their entirety: U.S. Pat. No. 4,874,368, U.S. Pat. No. 5,637,092, U.S. Pat. No. 5,782,073, U.S. Pat. No. 6,514,231, U.S. Pat. No. 6,824,016, U.S. Pat. No. 6,874,657, U.S. Pat. No. 6,972,005, U.S. Pat. No. 6,357,489, U.S. Pat. No. 6,568,434, WO 98/10703, WO 00/09074, and WO 07/059,801.

### SUMMARY

The invention relates to a system for delivery of fluids, the system comprising a syringe having an adjustable length plunger slidable within a respective syringe barrel.

Also, the invention relates to a multi-syringe system where a coupling element causes a plurality of plungers to slide in unison within their respective barrels to simultaneously dispense fluids therefrom. In the system, at least one of the plungers is a variable-length plunger that is selectively engagable to a coupling element.

It is now disclosed a system for delivery of fluids comprising: a. a syringe assembly comprising like-oriented first and second syringes to define a dispensing direction, each syringe having a respective plunger slidable within a respective syringe barrel, the plunger of the second syringe having an adjustable length; and b. a coupling element attached to and mechanically engaged to the plunger of the first syringe so that: i. lengthening or shortening of the adjustable-length plunger of the second syringe respectively causes the adjustable-length plunger of the second syringe to mechanically engage to or disengage from the coupling element; and ii. motion of coupling element in the dispensing direction causes each mechanically engaged plunger to longitudinally slide within its respective syringe barrel to move in unison with the coupling element.

It is now disclosed a system for delivery of fluids comprising: a. a syringe assembly comprising like-oriented first and second syringes to define a dispensing direction, each syringe having a respective plunger slidable within a respective syringe barrel; and b. a coupling element attached to and mechanically engaged to the plunger of the first syringe, the plunger of the second syringe having adjustable length and being selectively engagable to the coupling element such that: i. when the system is in a first configuration, there is clearance between the plunger of the second syringe and the coupling element so that the plunger of the second syringe is disengaged from the coupling element; ii. when the system is in a second configuration, plungers of the first and second syringes are simultaneously engaged to the coupling element and motion of coupling element in the dispensing direction causes each engaged plunger to longitudinally slide within its respective syringe barrel to move in unison with the coupling element.

In some embodiments, when the system is in the first configuration, the clearance between the plunger of the second syringe and the coupling element is in a direction along a central axis of a barrel and/or a plunger of the second syringe.

In some embodiments, the coupling element is permanently attached to the plunger of the first syringe.

In some embodiments, the coupling element is integrally formed with and/or glued to the plunger of the first syringe.

In some embodiments, the coupling element is locked to the plunger of the first syringe.

In some embodiments, the coupling element is detachably attached to the plunger of the first syringe.

In some embodiments, the coupling element is clamped to the plunger of the first syringe.

In some embodiments i. the dispensing direction defined by the first and second syringes and ii. a primary direction of contact between respective contact surfaces of the coupling element and of the adjustable-length plunger when mechanically engaged to each other are like-oriented.

In some embodiments, the plunger of second syringe includes a screw mechanism configured to modify a length of the plunger of second syringe.

In some embodiments, the plunger of second syringe includes a screw mechanism configured to modify a length of the plunger of second syringe.

In some embodiments, rotation of a rotatable element around an axis parallel to and/or co-linear with a central axis of second syringe is operative to adjust a length of the adjustable-length plunger of the second syringe to engage to or disengage from the coupling element.

In some embodiments, the plunger of the second syringe comprises: i. an internally threaded sleeve; and ii. externally threaded shaft arranged within the internally threaded sleeve such that rotation of the shaft within the sleeve causes longitudinal motion of the shaft relative to the sleeve, thereby adjusting the length of the plunger of second syringe.

In some embodiments, the syringe assembly further comprises a third syringe that is like-oriented with the first and second syringes, a plunger of the third syringe being mechanically coupled to the coupling element such that motion thereof causes the plunger of the third syringe to longitudinally slide within its respective syringe barrel to move in unison with the coupling element.

In some embodiments, the coupling element is permanently attached to the plunger of the third syringe.

In some embodiments, the coupling element is integrally formed with and/or glued to the plunger of the third syringe.

In some embodiments, the coupling element is locked to the plunger of the third syringe.

In some embodiments, the coupling element is detachably attached to the plunger of the third syringe.

In some embodiments, the coupling element is clamped to the plunger of the third syringe.

In some embodiments, when a central axis of the third syringe substantially equidistant from central axes of the first and the third syringes.

In some embodiments, a cross-sectional area of the barrel of the first syringe and/or a cross-sectional area of the barrel the second syringe is equal to that of the third syringe.

In some embodiments, a cross-sectional area of the barrel of the first syringe and/or a cross-sectional area of the barrel the second syringe different from that of the third syringe.

In some embodiments, the system further comprises a fluid delivery catheter including first and second lumen embedded therein spanning substantially an entirety thereof and configured to respectively receive fluid components discharged from respective barrels of the first and third syringes to define separate channels of fluid component delivery to a tip of lumen.

In some embodiments, the system further comprises a e. a fluid discharge conduit configured to receive fluids discharged from the barrel of the second syringe, an outlet of the barrel of the first syringe being in fluid communication with an exit location of the fluid discharge conduit so that fluids exiting the barrel of the first syringe via fluid discharge conduit mix with fluids exiting the barrel of the second syringe en route to a proximal end of the first lumen within the fluid deliver catheter.

In some embodiments, the system further comprises a comprising a check valve configured to regulate flow through the fluid discharge conduit so as to substantially prevent of fluids from the outlet thereof back into barrel of the second syringe.

In some embodiments, a ratio between the longer and shorter lengths of the plunger of the second syringe is at least 1.05 or at least 1.1 or at least 1.15 or at least 1.2 or at least 1.25 or at least 1.3.

In some embodiments, a length difference between the longer and shorter lengths is at least 1 cm.

In some embodiments, a difference between the longer and shorter lengths is at least 5% or at least 10% or at least 15% or at least 20% or at least 25% or at least 30% of an internal length of the barrel of the second syringe.

In some embodiments, the coupling element has a recess dimensioned to match a protruding portion of the second syringe plunger located at a proximal portion thereof.

In some embodiments, a cross-sectional area of barrel of the first syringe is equal to that of the second syringe.

In some embodiments, across-section-area of a barrel of the first syringe differs from that of the second syringe.

In some embodiments, a cross-sectional area of barrel of the first syringe is equal to that of the second syringe.

In some embodiments, the system further comprises: c. a fluid discharge conduit configured to receive fluids discharged from the barrel of the second syringe, an outlet of the barrel of the first syringe being in fluid communication with an exit location of the fluid discharge conduit so that fluids exiting the barrel of the second syringe via fluid discharge conduit mix with fluids exiting the barrel of the first syringe.

In some embodiments, the system further comprises a check valve configured to regulate flow through the fluid discharge conduit so as to substantially prevent of fluids from the outlet thereof back into barrel of the second syringe.

In some embodiments, the system further comprises a fluid delivery catheter including at least one lumen located therein operative to receive the mixture of fluids from the barrels of the first and second syringes.

In some embodiments, the system further comprising: a spike cup including a spike cup conduit therein between a bottom of the spike cup and an interior location within the spike cup, an upper end of the conduit being a sharp end for puncturing a septum of a vial reservoir containing a loadable fluid; a loading port directly or indirectly attached to a given barrel of one of the syringes and configured to receive a lower end of the spike cup conduit so that when engaged thereof fluid flows through the spike cup conduit into the given barrel so as to load the given barrel.

In some embodiments, the loading port is rotatable between open and closed configurations such that only when in the open configuration is the loading port is open to receive fluid therein.

It is now disclosed a method for delivery of fluids, the system comprising: a. providing a syringe assembly comprising: i. like-oriented first and second syringes to define a dispensing direction, each syringe having a respective plunger slidable within a respective syringe barrel, the plunger of the second syringe having an adjustable length; and ii. a coupling element attached to and mechanically engaged to the plunger of the first syringe b. lengthening or shortening of the adjustable-length plunger of the second syringe so as to causes the adjustable-length plunger of the second syringe to mechanically engage to or disengage from the coupling element; and c. moving coupling element in the dispensing direction so as to cause each mechanically engaged plunger to longitudinally slide within its respective syringe barrel to move in unison with the coupling element.

### BRIEF DESCRIPTION OF THE DRAWINGS

FIGS. 1A-1B, 2A-2B illustrate a multi-syringe system including a selectively-engagable syringe plunger that is decoupled from a coupling element.

FIGS. 3A-3B, 4A-4B illustrate the same system when selectively-engagable syringe plunger that is coupled to the coupling element.

FIG. 5 illustrates a method of operating the same system.

FIGS. 6A-6B illustrate a first variable-length plunger.

FIG. 7A illustrate a proximal end of a plunger when disengaged from the coupling element.

FIG. 7B illustrate a proximal end of a plunger when engaged to the coupling element.

FIG. 8 illustrates a technique for loading the system.

### DETAILED DESCRIPTION OF EMBODIMENTS

Embodiments of the present invention relate to a multi-syringe system/device providing multiple operating modes. When the device is in a first operating mode, N (N is a positive integer equal to two or more—i.e. N≧2) like-oriented syringes are coupled together so that motion of a so-called coupling element in a dispensing direction causes multiple plungers to move in unison within each of their respective barrels so that fluid components are simultaneously dispensed from each barrel of the N syringes. The coupling element connects the multiple plungers to each other so that plungers within all N barrels slide together in unison.

When in a second operating mode, motion of the coupling element in the dispensing direction causes plunger(s) to move in unison within only M barrels where M is a positive integer less than N (i.e. 1≦M<N). Thus, in the second operating mode, motion of the coupling element causes fluid components to be dispensed from respective barrel(s) of M syringe(s).

In one non-limiting example, a single multi-syringe device may be operated, at the user's discretion either in an N-component mode to simultaneously administer N components or in M-component mode to simultaneously administer M components.

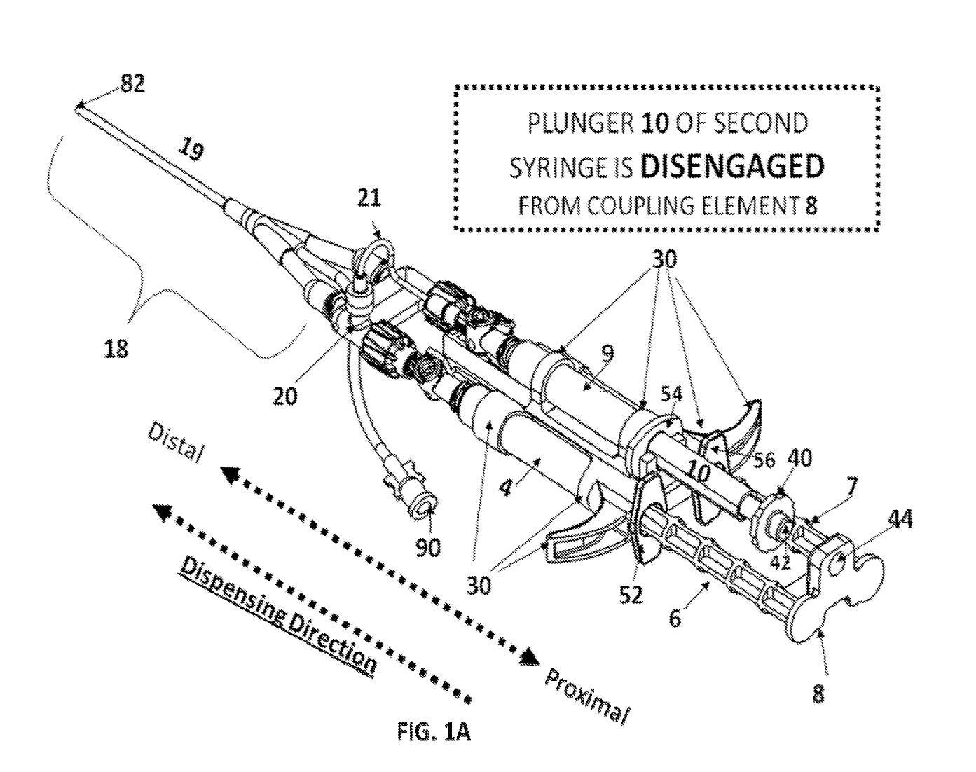

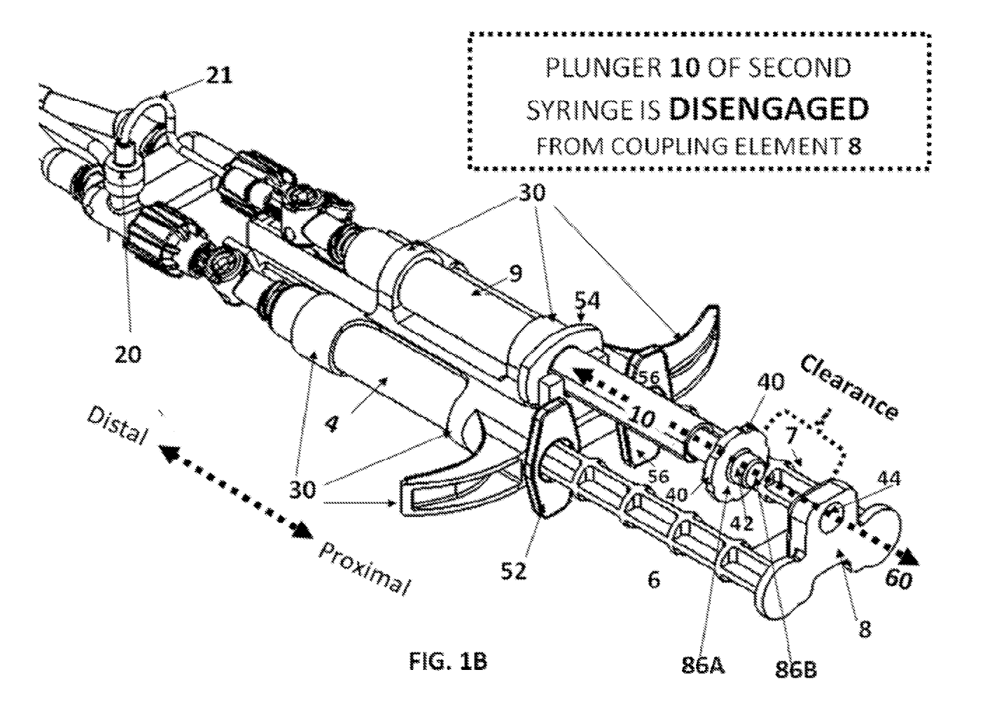

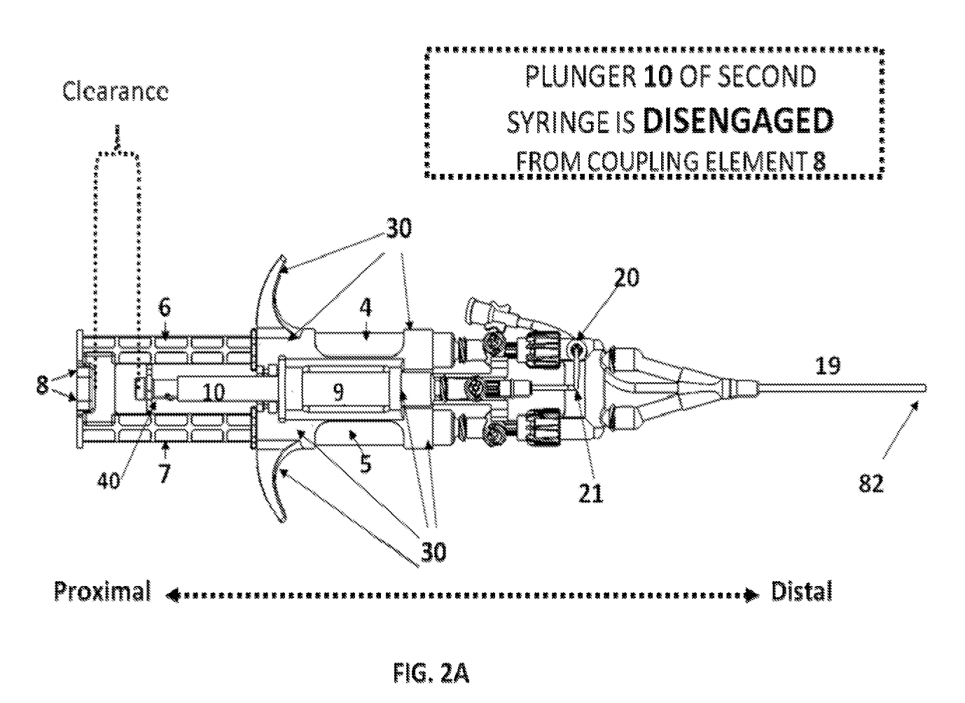

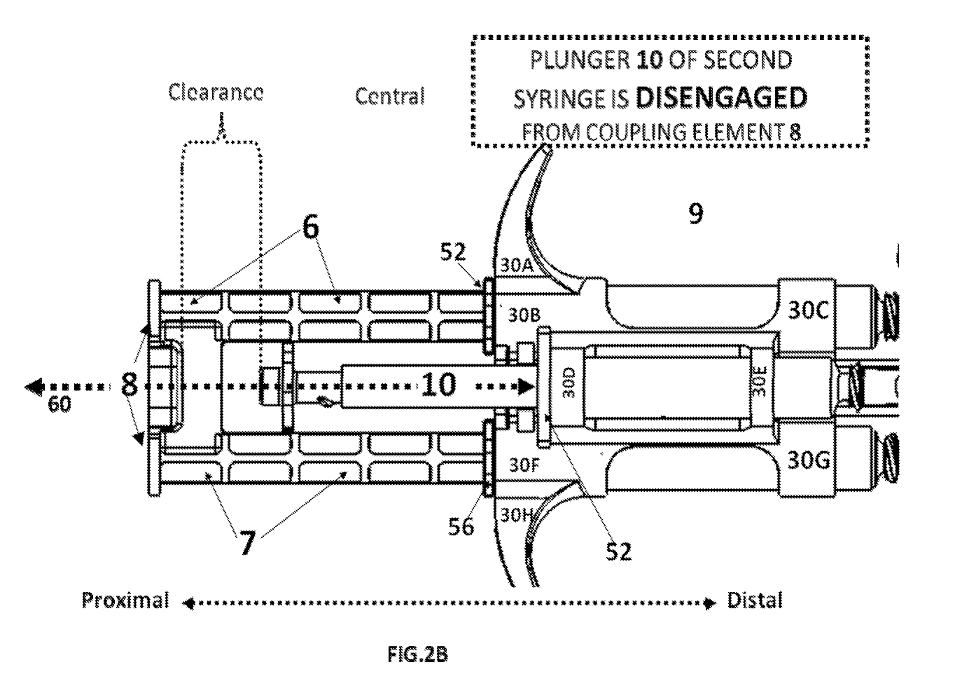

FIGS. 1-4 illustrate a specific multi-plunger device where N=3 and M=2 including three like-oriented syringes. In the non-limiting example of FIGS. 1-4, the multi-syringe device includes three syringes, two of which have plungers 6, 7 that are permanently attached to coupling element 8. In FIG. 1A, first and second syringes respectively having barrels 4,9 are visible. In FIG. 2A, a barrel 5 of a third syringe within which a third plunger 7 slides is visible.

Because plungers 6,7 of the first and third syringes are permanently attached to coupling element 8, in general, motion of coupling element 8 in a dispensing direction causes plungers 6, 7 to move in unison with each other and with coupling element 8 respectively within barrels 4,5 to simultaneously dispense fluid therefrom.

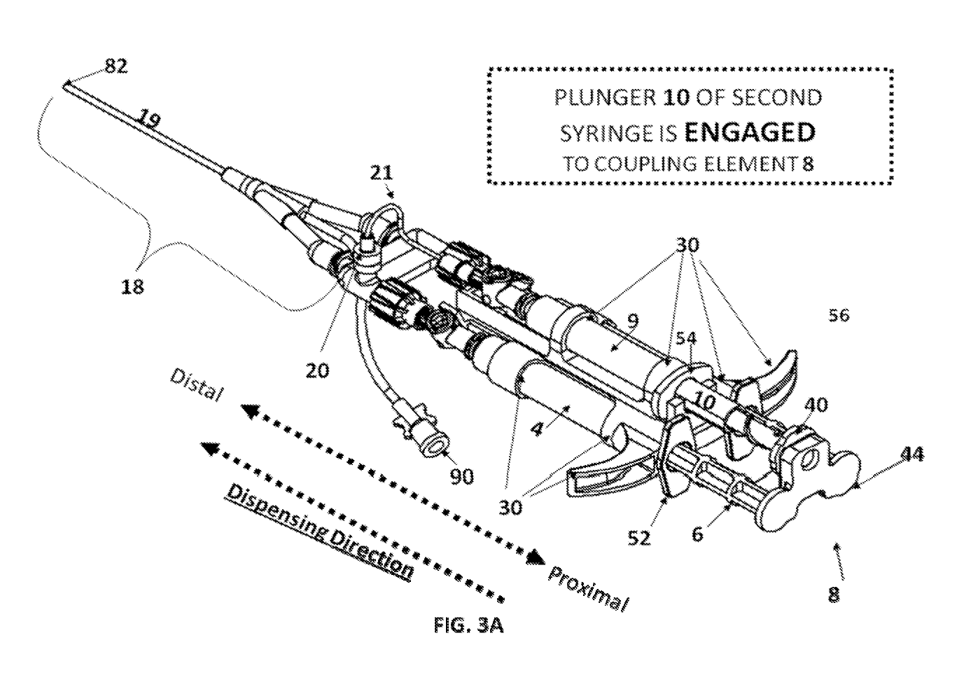

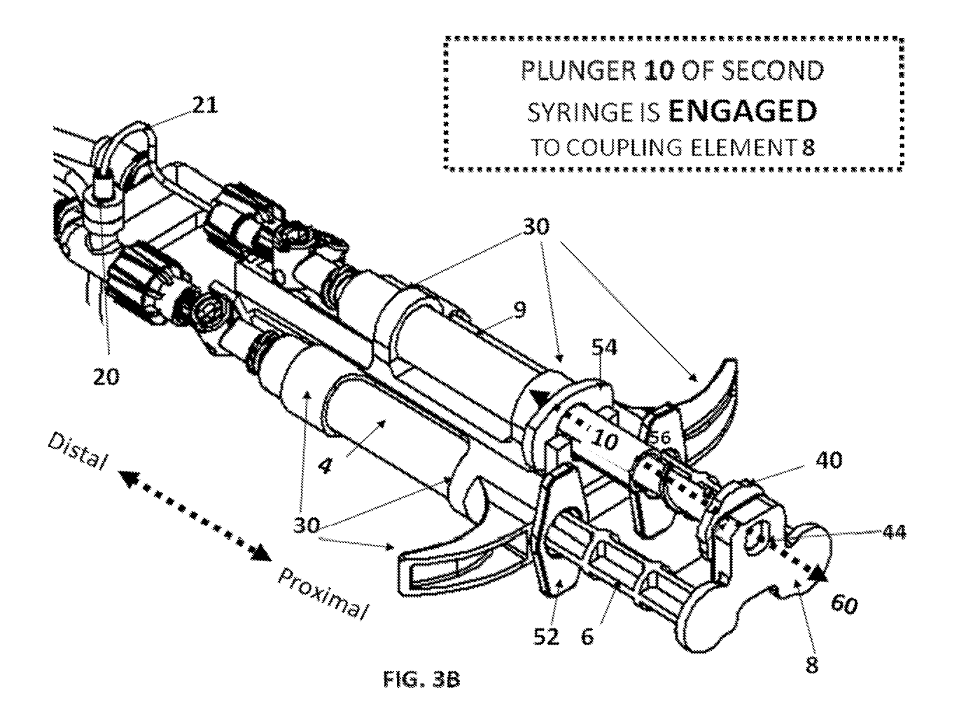

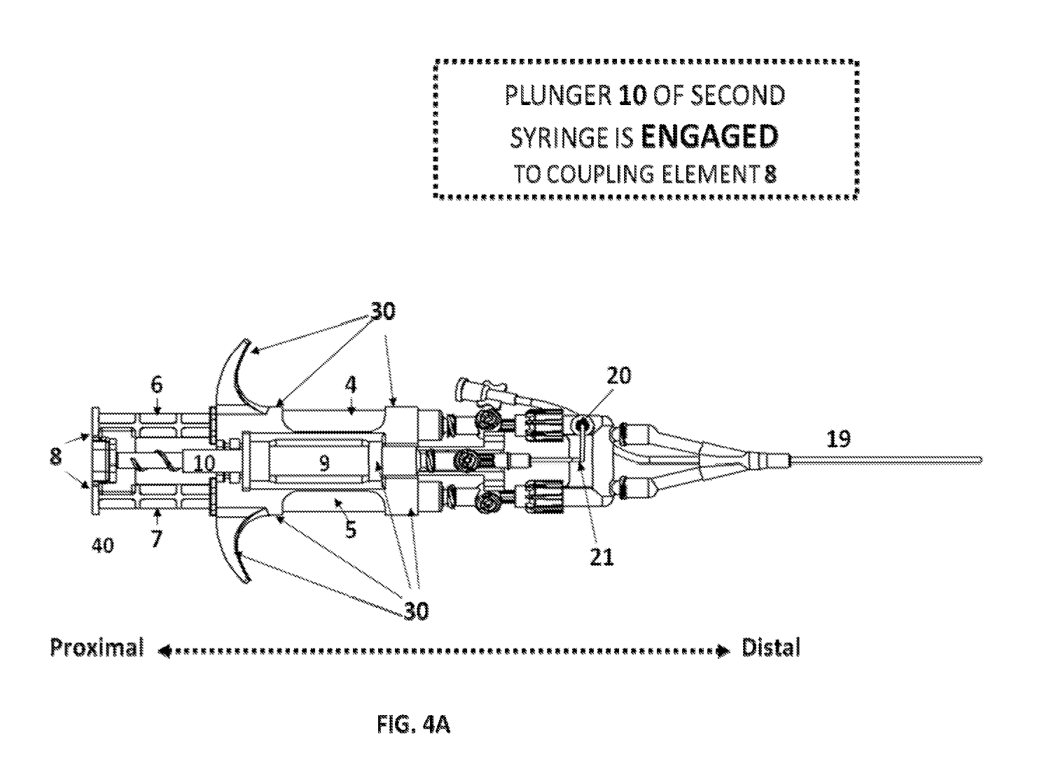

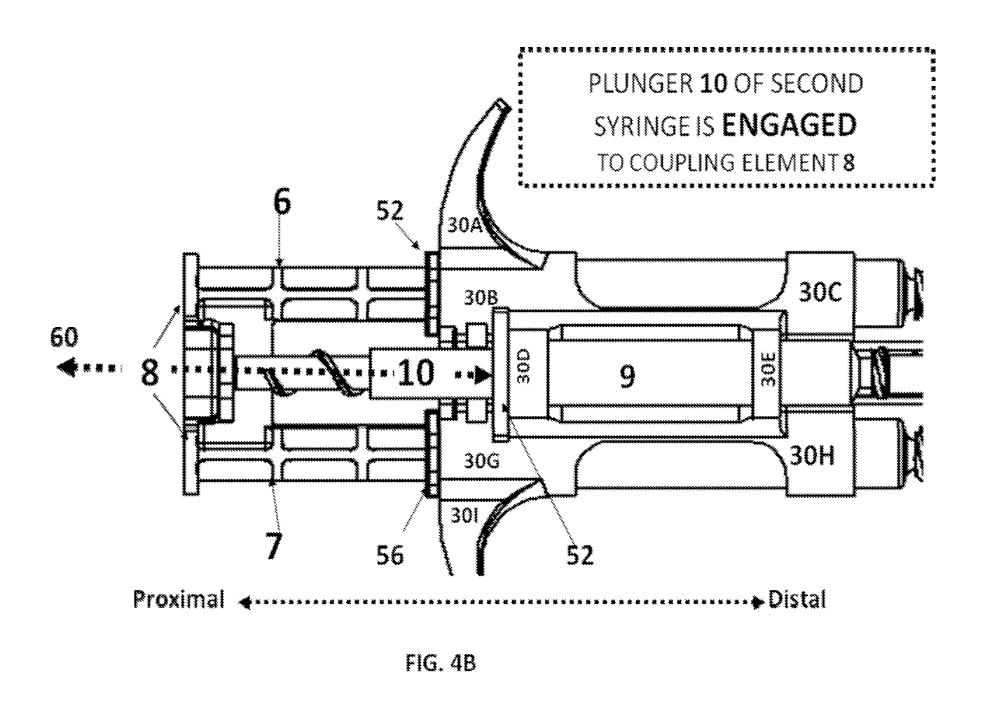

One salient feature of the multi-syringe device is that plunger 10 (i.e. arranged within the barrel 9 of the second syringe) is selectively couplable/engable to coupling element 8. When plunger 10 is coupled/engaged as in FIGS. 3-4, motion of coupling element 8 in a dispensing direction causes selectively couplable/engable plunger 10 to move in unison with plungers 6, 7. Simultaneous motion of plungers 6, 7,10 within respective barrels 4, 5, 9 causes fluid to be simultaneously expelled or dispensed from all three syringe barrels 4,5,9.

As will be discussed below, in some embodiments, plunger 10 is a variable length plunger—lengthening of variable-length plunger 10 is operative to bring a proximal end thereof into contact with coupling element 8 to mechanically engage thereto.

The like-oriented syringes are all substantially parallel to each other so as to define a dispensing direction—as illustrated in FIG. 1A, the dispensing direction is co-oriented with a ‘proximal-distal vector’ orientation.

FIGS. 1-2 illustrate the multi-syringe device when in ‘two-component’ operating mode. When in two-component mode, coupling element 8 is disengaged from selectively couplable plunger 10, and coupled to permanently-attached plungers 6, 7. When in two-component mode, a single stroke of coupling element 8 causes plungers 6, 7 to simultaneously slide within respective syringe barrels 4,5 so that fluids are simultaneously expelled therefrom. In one use case, it is possible to simultaneously dispense a fibrinogen component from barrel 4 and a thrombin component from barrel 5—for example, so that upon separately exiting barrels 4,5 each component separately enters into respective lumens within catheter 19 and exits a distal end 82 thereof. In this use case, the components (i.e. fibrinogen and the thrombin) may only mix with each other after exiting catheter 19.

Also illustrated in FIGS. 1B, 2A and 2B (i.e. relating to the ‘two-component’ operating mode) is a ‘clearance’ between (i) a proximal end of plunger 10 and (ii) coupling element 8. When the multi-syringe device is in the ‘two-component’ operating mode of FIGS. 1-2, motion along the ‘proximal-distal’ axis or ‘dispensing’ axis of coupling element 8 causes in-tandem motion of the permanently attached plungers 6, 7 without setting plunger 10 in motion—fluid is thus expelled from barrels 4,5 and not from barrel 9.

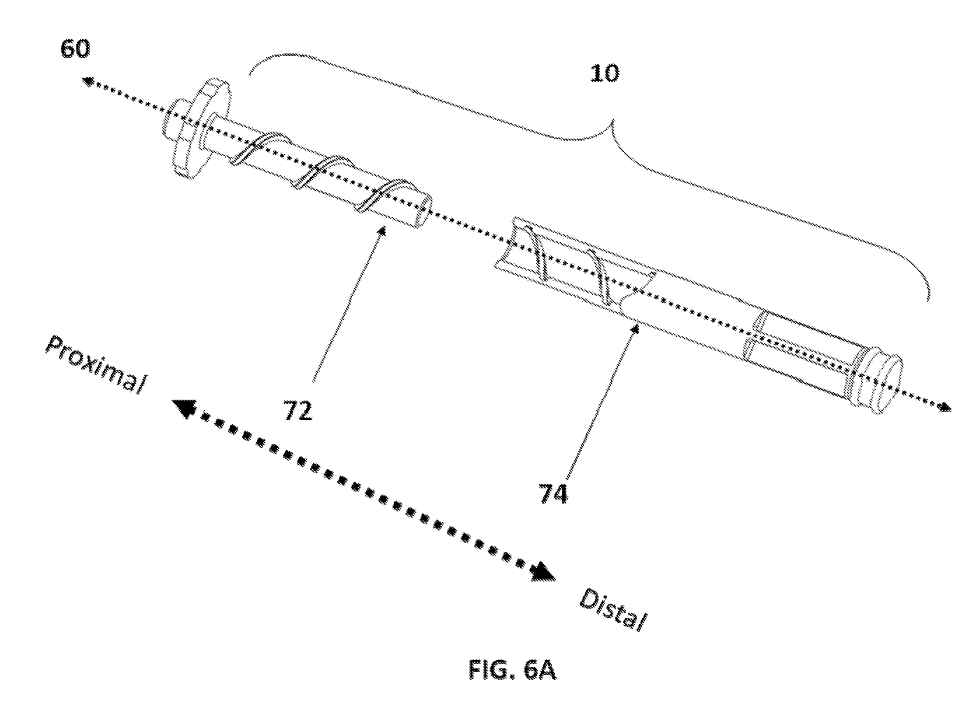

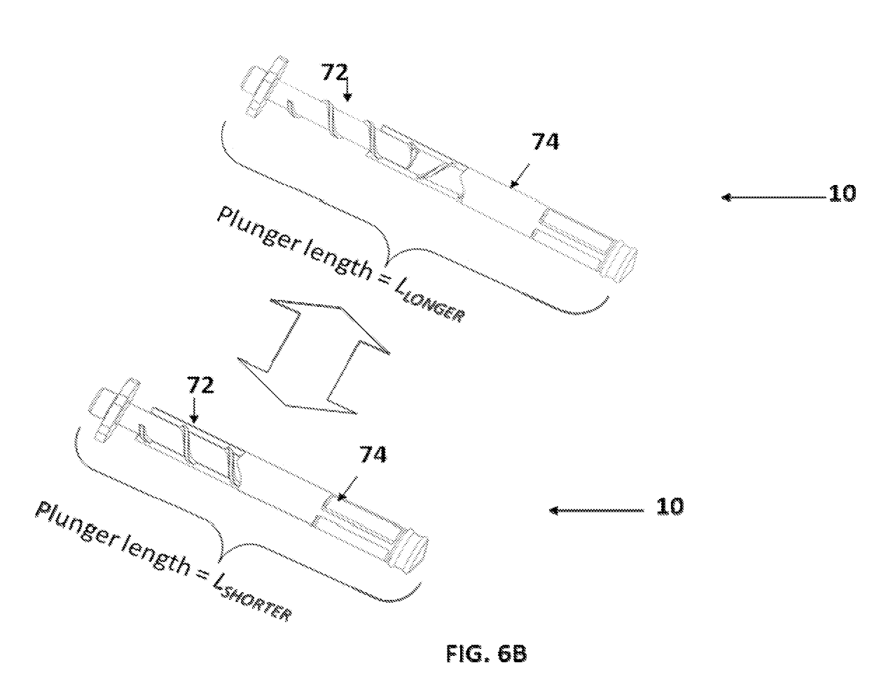

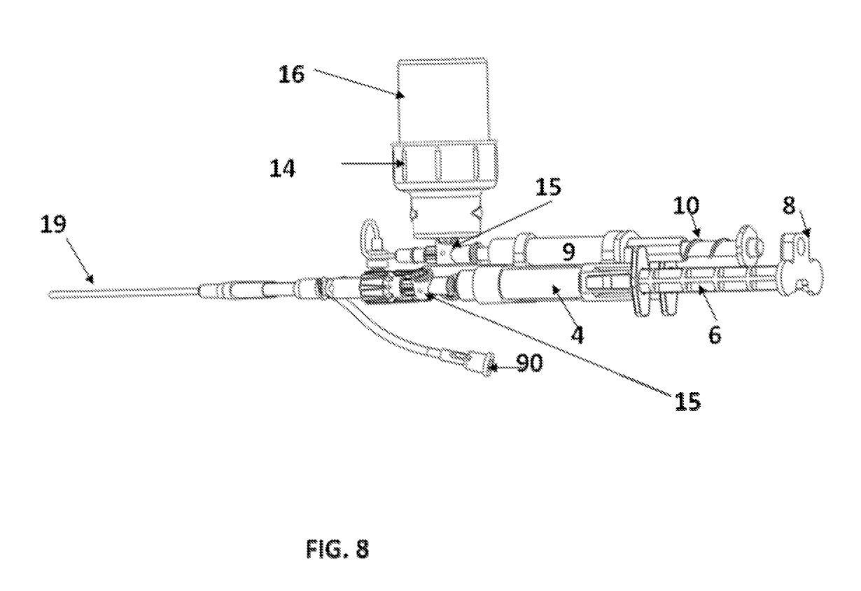

FIG. 3-4 describe the multi-syringe device in the ‘three-component’ operating mode. In the non-limiting examples of FIGS. 1-5, plunger 10 of multi-syringe device is a so-called ‘variable length plunger’—when the plunger length is ‘shorter’ multi-syringe device is in two-component mode, discussed above. In order to transition from two-component mode to three-component mode, it is possible to increase a length of variable-length plunger—non-limiting examples of accomplishing this are discussed below with reference to FIGS. 6 and 8.

When in three-component mode, coupling element 8 is in contact with a proximal location on plunger 10 so that plunger 10 may be said to be ‘coupled to’ coupling element 8. In this mode, motion of coupling element 8 in the dispensing direction causes sliding motion of three plungers 6, 7,10 within respective barrels 4, 5, 9 so that fluid components are respectively and simultaneously expelled from each of the three barrels 4,5, and 9. In this mode, coupling element 8 pushes upon plunger 10 to force plunger 10 to slide within its barrel 9.

In one non-limiting use case, (i) when the multi-syringe device is in two component mode, only fibrinogen and thrombin components are administered to a patient and (ii) when the multi-syringe device is in three component mode, it is possible to simultaneously administer fibrinogen (i.e. from barrel 4), thrombin (i.e. from barrel 5) and a supplement (e.g. antibiotics, anti-inflammatory agents, chemotherapy agents, growth factors, anti-cancer drugs analgesics, proteins, hormones, antioxidants and the like) from barrel 9. The same multi-syringe device may thus be employed, at the user's discretion, in either two-component mode or three-component mode, allowing a practitioner (e.g. a surgeon) to utilize the same multi-syringe device for both purposes.

In one example, the fibrinogen component and thrombin component can be administered simultaneously and the supplement may be administered at any time during the administration of the two components, at the user's discretion. Thus, at one time point during the administration, the user may decide to administer the supplement along with the fibrinogen component and thrombin component whereas at another time point, the user may decide to administer only the two components without the supplement, meaning that some portion of the fibrin sealant will include the supplement while other portions will only consist fibrin sealant.

The step of engagement and disengagement of plunger 10 from coupling element 8 can be carried out alternately during the administration of the liquid components. For example, the administration can be initiated wherein plunger 10 is disengaged from coupling element 8 and multi-syringe device is in ‘two-component mode. In a second step, plunger 10 can be engaged with coupling element 8 followed by a third step of disengaging plunger 10 from coupling element 8.

The administration can be carried out by injection e.g. when a needle is installed on the dispensing end of the device, by dripping, or by spraying (e.g. when a gas inlet 90 e.g. a pressurized gas is added so that an inlet gas stream mixes with fluid dispensed from the syringe barrel(s)). The administration can also be carried out by casting the components into a mold.

One feature of the device illustrated in FIGS. 1-4 is that the diameters of barrels 4, 5, 9 are equal to each other—thus, movement of coupling element 8 in a dispensing direction causes equal volumes of fluid to be expelled respectively from each barrel 4, 5, 9. This is not a limitation. In other embodiments, the diameters of two or more of barrels 4, 5, 9 may differ from each other. It is possible to manufacture the device to control a ratio between diameters of two or more barrels according to a desired ratio between the fluid components to be dispensed therefrom.

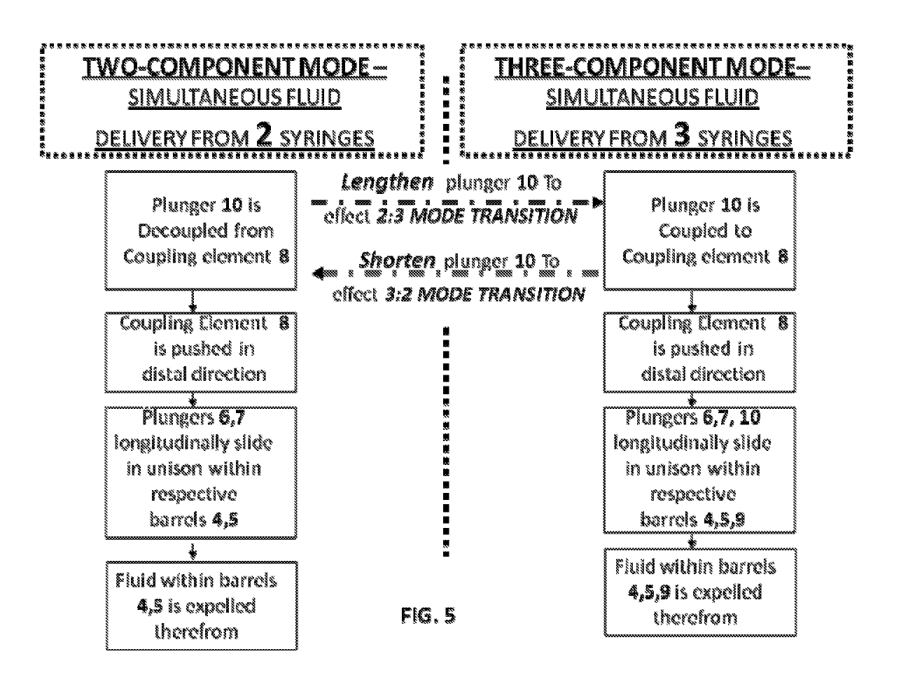

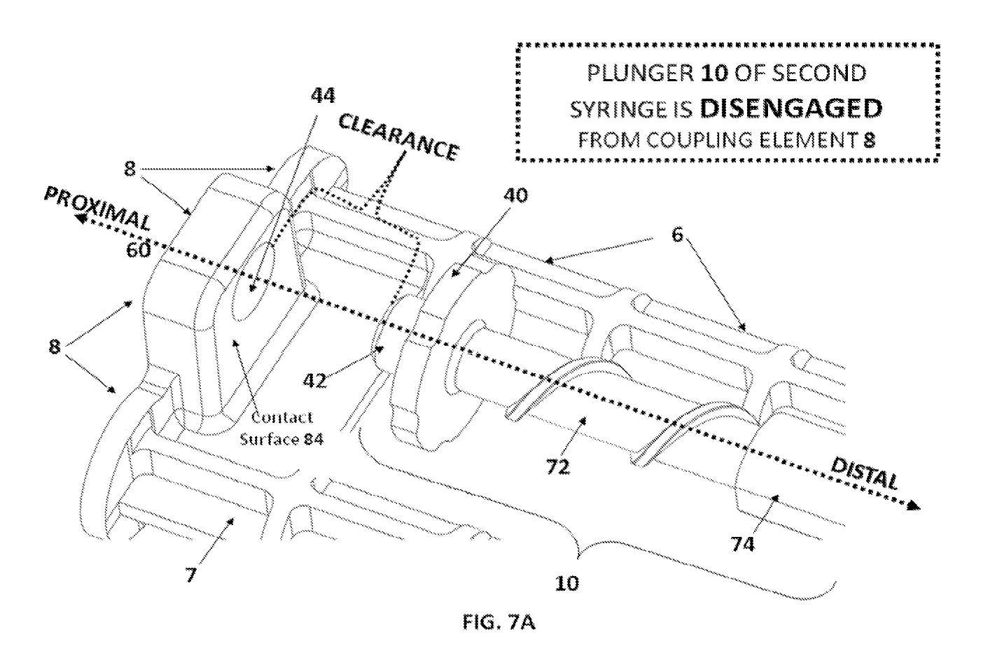

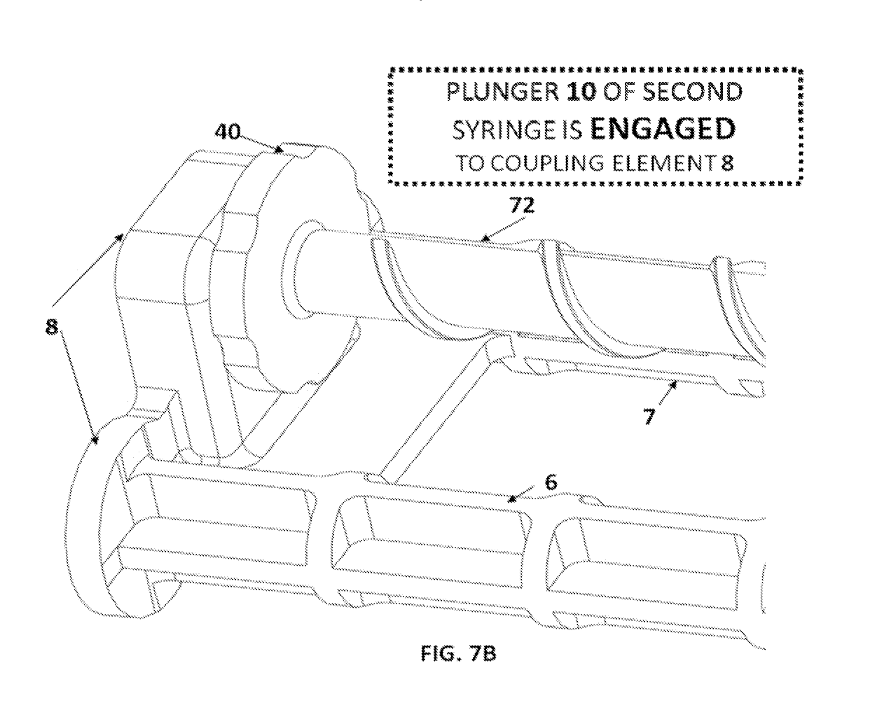

FIG. 5 is a flow chart of a technique of operating the multi-syringe device. As illustrated in FIG. 5, when plunger 10 is lengthened (i.e. sufficiently so that a proximal portion of the plunger 10 contacts a ‘distal-facing’ surface (see 84 of FIG. 7A) of coupling element 8), the multi-syringe device transitions from the ‘two-component mode’ to the ‘three-component’ mode. This is referred to as a 2:3 mode transition in FIG. 5. When the multi-syringe device is in ‘three-component mode’ and plunger 10 is shortened (i.e. to eliminate contact between coupling element 8 and plunger 10), the multi-syringe device transitions from ‘three-component mode’ to ‘two-component mode’—this is referred to as 3:2 mode transition in FIG. 5. As indicated in the figure, when in two-component mode, movement of coupling element 8 in a dispensing direction (i.e. substantially parallel to a ‘proximal-distal’ direction) causes two plungers 6, 7 to slide in unison within their respective barrels 4,5 and fluid to be expelled therefrom. When in three-component mode, movement of coupling element 8 in a dispensing direction additionally causes plunger 10 to slide in unison with plungers 6, 7 so that fluid is expelled simultaneously from three syringe barrels 4,5, 9.

Typically, coupling element 8 imparts momentum about plunger 10 by pushing plunger 10 without relying on friction or any other additional mechanism. The orientation of a ‘contact’ surface 84 of coupling element 8 in contact with plunger 10 is defined by its local normal. As shown in FIG. 7A, this contact surface faces the distal direction. The orientation of the ‘contact’ surface (see 86A and/or 86B of FIG. 1B) of plunger 10 in contact with coupling element 8 is in the opposite direction—i.e. facing the proximal direction.

Thus, a ‘contact direction’ from the coupling element to the adjustable-length plunger is substantially in the dispensing direction along the proximal-distal axis of the multi-syringe device. It may thus be said that the direction of contact from the coupling element 8 to plunger 10 is like-oriented with a dispensing direction. This allows coupling element 8 to push plunger 10 by applying a force along the contact direction, thereby causing plunger 10 to slide in a dispensing direction to expel or dispense fluid from a barrel 9 within which plunger 10 slides.

For the present disclosure, the term ‘fluid’ is defined broadly and may refer to any flowable matter including but not limited to liquids and flowable gels.

As discussed above, in some embodiments, plunger 10 may be a variable-length plunger. One example of a variable length plunger is illustrated in FIGS. 6A-6B. In this example, variable length plunger 10 includes an externally-threaded shaft 72 rotatable within an internally threadable sleeve 74 (this feature is shown in a broken out section in order to see its internal structure). Rotation of the shaft 72 around a central axis 60 of plunger 10 causes longitudinal motion of shaft 72 within sleeve 74 to modify a position of shaft 72 relative to sleeve 74, and lengthen or shorten variable-length plunger. The direction of rotation in FIG. 6 is just an example and other rotation mechanisms for lengthening or shortening a length of plunger 10 (for example, which require rotating around a rotation axis having a different orientation) are contemplated.

FIGS. 7A-7B illustrate a proximal region of the variable-length plunger of FIGS. 6A-6B in proximity with coupling element 8 at a time plunger 10 is disengaged from coupling element 8. As illustrated in FIG. 7A, there is a clearance between (i) a proximal portion of plunger 10; and (ii) a distal portion of coupling element 8. In FIG. 7B, after plunger 10 is lengthened, no such clearance exists—instead, there is contact between a distal-facing surface 84 of coupling element 8 and plunger 10. In the non-limiting example of FIG. 7, variable-length plunger 10 includes a shoulder 40 portion and a proximally-protruding element 42 protruding from shoulder 40 which is dimensioned to fit within recess 44 of coupling element 8.

Also illustrated in FIGS. 7A-7B are plungers 6, 7 which are permanently attached to coupling element 8. One example of ‘permanently attached’ plungers 6, 7 are plungers glued to coupling element 8. In another example, plungers 6, 7 are integrally formed with coupling element 8—this may reduce the number of parts required to manufacture the multi-syringe device. It is noted that the ‘permanently attached’ feature is not a requirement—in another example, plungers 6, 7 are detachably attached to coupling element 8—for example, by a snap or any other mechanism.

Additional elements illustrated in FIGS. 1-4 are now discussed. Multi-syringe device includes manifold 18 through which fluids delivered from syringe barrels 4,5,9 pass to exit the device. In some embodiments, manifold 18 includes catheter 19 having multiple lumen to provide separate flow ‘channels’. Fluids components (e.g. fibrinogen and thrombin) expelled from syringe barrels 4,5 subsequently and separately flow through catheter 19 and exit distal end 82 thereof so as to be dispensed separately—this may be useful for ‘surgical glue’ applications.

In the example of FIGS. 1-4, fluids expelled from barrel 9 may be introduced into a flow path between an outlet of syringe barrel 4 and a proximal end of catheter 19 so as to mix with each other before entering “fluid delivery” catheter 19. For example, fluid expelled from barrel 9 flows through fluid discharge conduit 21 and one-way valve 20 or ‘check valve’ into a location along the aforementioned flow path. One-way valve 20 prevents “back-flow” of a fluid component exiting barrel 4 from flowing into barrel 9.

Also visible in FIGS. 1-4 are the following elements: (i) attachment/holding element or brace 30 which holds the first, second and third syringes together in the same general orientation; (ii) shoulders 52, 54, 56 of the first, second and third syringes; and (iii) central axis 60 of the barrel 9 of the second syringe and/or the plunger 10 therein. Attachment/holding element 30 includes various portions 30A-30H labeled in FIG. 2B.

As noted above, FIGS. 6A-6B describe a first mechanism for varying a plunger length 10. In some embodiments, the ‘rotation/screw’ mechanism provides an ease-of-use and may require fewer parts when manufacturing the multi-syringe device. The mechanisms for lengthening or shortening the plunger described herein is an example—the skilled artisan will recognize that various mechanisms may be employed to accomplish this task.

In the examples of FIGS. 1-7 one or more of the plungers is reversibly or ‘selectively’ engagable to coupling element. The mechanism for providing this feature includes a variable length plunger.

FIG. 8 relate to techniques for loading fluids into the multi-syringe device in accordance with some embodiments.

Prior to use, the barrels 4,5,9 are loaded with the liquid/fluid components. Loading of the barrels can be carried out by installing a spike cup 14 on a fluid control device/loading port 15, and placing a vial/reservoir 16 within the spike cup 14. The spike cup may comprise a protruding needle preferably adapted to puncture the vial's septum. The vial in the spike cup is punctured by the protruding needle, enabling liquid flow from the vial and into the barrel via the needle by drawing the plunger. The plunger is pulled in the opposite direction to the dispensing direction, resulting in drawing of the fluid component from the vial 16 into the barrel. In one example, plungers 6 and 7 (which are connected with each other, e.g. via part 8) are pulled simultaneously leading to loading of barrels 4 and 5. Typically, plunger 10 is pulled separately leading to loading of barrel 9.

FIG. 8 shows the upper part of the fluid control device corresponding to each syringe, revealing the connection interface having a designated structure for connecting with the spike cup. A spike cup 14 and a vial 16 connected to the fluid control device 15 of the second syringe is shown in FIG. 10.

Following loading of the barrels with the liquid components, the spike cup 14 and the vial 16 are removed by rotating the spike cup, thus allowing, in a subsequent step, dispensing of the liquid components from the barrels.

The structure and function of the fluid control device and its use for loading the barrels of the device with the fluid components as well as for dispensing the fluid components are explained in WO98/10703.

In the description and claims of the present application, each of the verbs, “comprise” “include” and “have”, and conjugates thereof, are used to indicate that the object or objects of the verb are not necessarily a complete listing of members, components, elements or parts of the subject or subjects of the verb.

The present invention has been described using detailed descriptions of embodiments thereof that are provided by way of example and are not intended to limit the scope of the invention. The described embodiments comprise different features, not all of which are required in all embodiments of the invention. Some embodiments of the present invention utilize only some of the features or possible combinations of the features. Variations of embodiments of the present invention that are described and embodiments of the present invention comprising different combinations of features noted in the described embodiments will occur to persons of the art.

What is claimed is:

1. A system for delivery of fluids, the system comprising:
a. a syringe assembly comprising like-oriented first and second syringes to define a dispensing direction, each syringe having a respective plunger slidable within a respective syringe barrel, the plunger of the second syringe having an adjustable length; and
b. a coupling element attached to and mechanically engaged to the plunger of the first syringe so that:
i. lengthening or shortening of the adjustable-length plunger of the second syringe respectively causes the adjustable-length plunger of the second syringe to mechanically engage to or disengage from the coupling element; and
ii. motion of the coupling element in the dispensing direction causes each mechanically engaged plunger to longitudinally slide within its respective syringe barrel to move in unison with the coupling element.

2. A system for delivery of fluids, the system comprising:
a. a syringe assembly comprising like-oriented first and second syringes to define a dispensing direction, each syringe having a respective plunger slidable within a respective syringe barrel; and
b. a coupling element attached to and mechanically engaged to the plunger of the first syringe, the plunger of the second syringe having adjustable length and being selectively engagable to the coupling element such that:
i. when the system is in a first configuration, there is clearance between the plunger of the second syringe and the coupling element so that the plunger of the second syringe is disengaged from the coupling element;
ii. when the system is in a second configuration, plungers of the first and second syringes are simultaneously engaged to the coupling element and motion of coupling element in the dispensing direction causes each engaged plunger to longitudinally slide within its respective syringe barrel to move in unison with the coupling element.

3. The fluid delivery system of claim 2 wherein when the system is in the first configuration, the clearance between the plunger of the second syringe and the coupling element is in a direction along a central axis of a barrel and/or a plunger of the second syringe.

4. The fluid delivery system of claim 1 or 2 wherein the coupling element is permanently attached to the plunger of the first syringe.

5. The fluid delivery system of claim 1 or 2 wherein the coupling element is integrally formed with and/or glued to the plunger of the first syringe.

6. The fluid delivery system of claim 1 or 2 wherein the coupling element is locked to the plunger of the first syringe.

7. The fluid delivery system of claim 1 or 2 wherein the coupling element is detachably attached to the plunger of the first syringe.

8. The fluid delivery system of claim 1 or 2 wherein the coupling element is clamped to the plunger of the first syringe.

9. The fluid delivery system of claim 1 or 2 wherein:
i. the dispensing direction defined by the first and second syringes and
ii. a primary direction of contact between respective contact surfaces of the coupling element and of the adjustable-length plunger when mechanically engaged to each other, are like-oriented.

10. The fluid delivery system of claim 1 or 2 wherein the plunger of second syringe comprises a screw mechanism configured to modify a length of the plunger of the second syringe.

11. The fluid delivery system of claim 1 or 2 wherein rotation of a rotatable element around an axis parallel to and/or co-linear with a central axis of second syringe is operative to adjust a length of the adjustable-length plunger of the second syringe to engage to or disengage from the coupling element.

12. The fluid delivery system of claim 1 or 2 wherein the plunger of the second syringe comprises:
i. an internally threaded sleeve; and
ii. externally threaded shaft arranged within the internally threaded sleeve such that rotation of the shaft within the sleeve causes longitudinal motion of the shaft relative to the sleeve, thereby adjusting the length of the plunger of second syringe.

13. The fluid delivery system of claim 1 or 2 wherein the syringe assembly further comprises a third syringe that is like-oriented with the first and second syringes, a plunger of the third syringe being mechanically coupled to the coupling element such that motion thereof causes the plunger of the third syringe to longitudinally slide within its respective syringe barrel to move in unison with the coupling element.

14. The fluid delivery system of claim 13 wherein the coupling element is permanently attached to the plunger of the third syringe.

15. The fluid delivery system of claim 13 wherein the coupling element is integrally formed with and/or glued to the plunger of the third syringe.

16. The fluid delivery system of claim 13 wherein the coupling element is locked to the plunger of the third syringe.

17. The fluid delivery system of claim 13 wherein the coupling element is detachably attached to the plunger 7 of the third syringe.

18. The fluid delivery system of claim 13 wherein the coupling element is clamped to the plunger 7 of the third syringe.

19. The fluid delivery system of claim 13 wherein when a central axis of the second syringe substantially equidistant from central axes of the first and the third syringes.

20. The fluid delivery system of claim 13 wherein a cross-sectional area of the barrel of the first syringe and/or a cross-sectional area of the barrel of the second syringe is equal to a cross sectional area of the barrel of the third syringe.

21. The fluid delivery system of claim 13 wherein a cross-sectional area of the barrel of the first syringe and/or a cross-sectional area of the barrel of the second syringe is different from a cross sectional area of the barrel of the third syringe.

22. The fluid delivery system of claim 13 further comprising a fluid delivery catheter comprising a first and second lumen embedded therein spanning substantially an entirety thereof and configured to respectively receive fluid components discharged from respective barrels of the first and third syringes to define separate channels of fluid component delivery to a tip of the catheter.

23. The fluid delivery system of claim 22 further comprising:
c. a fluid discharge conduit configured to receive fluids discharged from the barrel of the second syringe, an outlet of the barrel of the first syringe being in fluid communication with an exit location of the fluid discharge conduit so that fluids exiting the barrel of the second syringe via fluid discharge conduit mix with fluids exiting the barrel of the first syringe en route to a proximal end of the first lumen within the fluid delivery catheter.

24. The fluid system of claim 23 further comprising a check valve configured to regulate flow through the fluid discharge conduit so as to substantially prevent of fluids from the outlet thereof back into the barrel of the second syringe.

25. The fluid delivery system of claim 1 or 2 wherein a ratio between the longer and shorter lengths of the plunger of the second syringe is at least 1.2.

26. The fluid delivery system of claim 1 or 2 wherein a length difference between the longer and shorter lengths is at least 1 cm.

27. The fluid delivery system of claim 1 or 2 wherein a difference between the longer and shorter lengths is at least 15% of an internal length of the barrel of the second syringe.

28. The fluid delivery system of claim 1 or 2 wherein the coupling element has a recess dimensioned to match a protruding portion of the second syringe plunger located at a proximal portion thereof.

29. The fluid delivery system of claim 1 or 2 wherein a cross-sectional area of the barrel of the first syringe is equal to a cross-sectional area of the barrel of the second syringe.

30. The fluid delivery system of claim 1 or 2 wherein a cross-section-area of a barrel of the first syringe differs from a cross-sectional area of the barrel of the second syringe.

31. The fluid delivery system of claim 1 or 2 further comprising:
c. a fluid discharge conduit configured to receive fluids discharged from the barrel of the second syringe, an outlet of the barrel of the first syringe being in fluid communication with an exit location of the fluid discharge conduit so that fluids exiting the barrel of the second syringe via fluid discharge conduit mix with fluids exiting the barrel of the first syringe.

32. The fluid system of claim 31 further comprising a check valve configured to regulate flow through the fluid discharge conduit so as to substantially prevent of fluids from the outlet thereof back into barrel of the second syringe.

33. The fluid delivery system of claim 31 further comprising a fluid delivery catheter comprising at least one lumen located therein operative to receive the mixture of fluids from the barrels of the first and second syringes.

34. The fluid delivery device of claim 1 or 2 further comprising:
a spike cup including a spike cup conduit therein between a bottom of the spike cup and an interior location within the spike cup, an upper end of the conduit being a sharp end for puncturing a septum of a vial reservoir containing a loadable fluid;
a loading port directly or indirectly attached to a given barrel of one of the syringes and configured to receive a lower end of the spike cup conduit so that when engaged thereof fluid flows through the spike cup conduit into the given barrel so as to load the given barrel.

35. The system of claim 34 wherein the loading port is rotatable to open and close configurations such that only when in the open configuration the loading port is open to receive fluid therein.

36. A system for delivery of fluids, the system comprising:
a syringe having an adjustable length plunger slidable within a respective syringe barrel.

37. A method for delivery of fluids comprising:
providing a syringe assembly comprising:
i. like-oriented first and second syringes to define a dispensing direction, each syringe having a respective plunger slidable within a respective syringe barrel the plunger of the second syringe having an adjustable length; and
ii. a coupling element attached to and mechanically engaged to the plunger of the first syringe
lengthening or shortening of the adjustable-length plunger of the second syringe so as to causes the adjustable-length plunger of the second syringe to mechanically engage to or disengage from the coupling element; and
moving coupling element in the dispensing direction so as to cause each engaged plunger to longitudinally slide within its respective syringe barrel to move in unison with the coupling element and thereby causing fluid to be simultaneously expelled from the barrels.
